(19) United States
(12) Patent Application Publication
(10) Pub. No.: US 2024/0242793 A1
(43) Pub. Date: Jul. 18, 2024

(54) MEDICAL SUPPORT SYSTEM, MEDICAL SUPPORT METHOD, MEDICAL INFORMATION PROCESSING APPARATUS, AND STORAGE MEDIUM

(71) Applicants: CANON MEDICAL SYSTEMS CORPORATION, Tochigi (JP); Canon Kabushiki Kaisha, Tokyo (JP)

(72) Inventors: Akihiro KOGA, Shinagawa (JP); Koji HIRATA, Kasukabe (JP); Kohei WATANABE, Utsunomiya (JP); Yasuo SAKURAI, Nasushiobara (JP); Katsuaki DEGUCHI, Otawara (JP)

(73) Assignees: CANON MEDICAL SYSTEMS CORPORATION, Tochigi (JP); Canon Kabushiki Kaisha, Tokyo (JP)

(21) Appl. No.: 18/411,534

(22) Filed: Jan. 12, 2024

(30) Foreign Application Priority Data
Jan. 13, 2023 (JP) 2023-003635

(51) Int. Cl. G16H 10/40; G16H 10/60; G16H 15/00; G16H 50/30

(52) U.S. Cl. CPC G16H 10/40; G16H 10/60; G16H 15/00; G16H 50/30

(57) ABSTRACT

A medical support system according to an embodiment includes processing circuitry and a display. The processing circuitry acquires biometric data of a subject to be examined or treated. The processing circuitry generates information on the probability of success in cell production in the future based on biological samples of the subject, on the basis of the biometric data. The display displays information on the probability of success in cell production in the future.

### CROSS-REFERENCE TO RELATED APPLICATIONS

This application is based upon and claims the benefit of priority from Japanese Patent Application No. 2023-003635, filed on Jan. 13, 2023; the entire contents of which are incorporated herein by reference.

### FIELD

Embodiments disclosed herein relate to a medical support system, a medical support method, a medical information processing apparatus, and a storage medium.

### BACKGROUND

In regenerative medicine (for example, organ regeneration using induced pluripotent stem (iPS) cells, or the like), allogeneic transplantation is performed to find a donor with a matching human leukocyte antigen (HLA) type and to use cells provided by the donor in order to reduce the risk of immune rejection. However, since there are certain limitations in reducing the risk of immune rejection with allogeneic transplantation, the implementation of regenerative medicine by autologous transplantation using patient's own cells (for example, blood or the like) is being considered as one solution for avoiding the risk of immune rejection.

### BRIEF DESCRIPTION OF THE DRAWINGS

FIG. 1 is a diagram illustrating an example of the configuration of a medical support system according to a first embodiment;

FIG. 2 is a diagram for explaining an overview of the medical support system according to the first embodiment;

FIG. 3A is a diagram for explaining the correlation between the stem cell content and the probability of iPS cell establishment according to the first embodiment;

FIG. 3B is a diagram for explaining the correlation between the stem cell content and the probability of iPS cell establishment according to the first embodiment;

FIG. 4 is a diagram illustrating an example of display information according to the first embodiment;

FIG. 5 is a flowchart showing an example of the processing procedure of a medical information processing apparatus according to the first embodiment;

FIG. 6 is a diagram for explaining an overview of a medical support system according to a second embodiment;

FIG. 7 is a diagram illustrating an example of display information according to the second embodiment; and

FIG. 8 is a flowchart showing an example of the processing procedure of a medical information processing apparatus according to the second embodiment.

### DETAILED DESCRIPTION

A medical support system according to an embodiment includes processing circuitry and a display. The processing circuitry is configured to acquire biometric data of a subject to be examined or treated. The processing circuitry is configured to generate information on the probability of success in cell production in the future based on biological samples of the subject, on the basis of the biometric data. The display is configured to display information on the probability of success in cell production in the future.

Embodiments of a medical support system, a medical support method, a medical information processing apparatus, and a storage medium are described in detail below with reference to the drawings. Note that the medical support system, the medical support method, the medical information processing apparatus, and the storage medium according to the present application are not limited by the following embodiments.

### First Embodiment

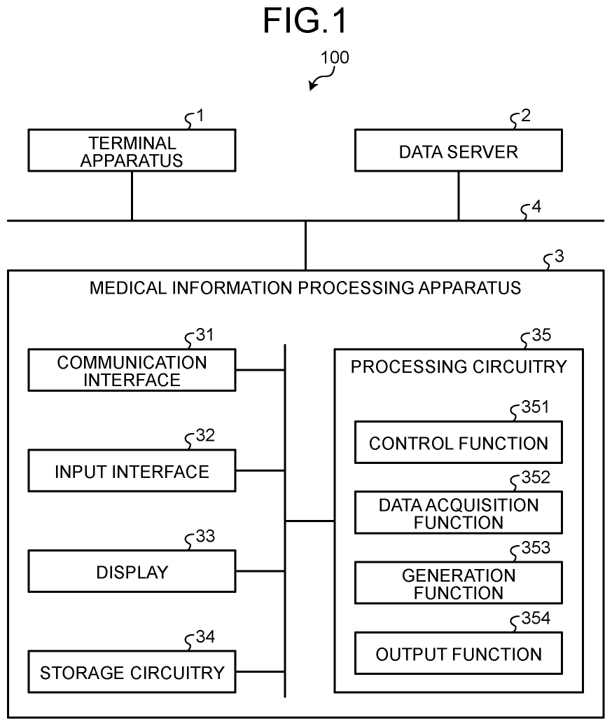

FIG. 1 is a diagram illustrating an example of the configuration of a medical support system 100 according to a first embodiment. For example, as illustrated in FIG. 1, the medical support system 100 according to the present embodiment includes a terminal apparatus 1, a data server 2, and a medical information processing apparatus 3 that are communicably connected to one another via a network 4. The network 4 includes, for example, a local area network (LAN) and a wide area network (WAN). Note that various other devices and systems may be connected to the network 4 illustrated in FIG. 1.

The terminal apparatus 1 is an apparatus (first terminal) used by medical personnel, such as doctors and technicians working in a hospital, and subjects to be examined or treated, in order to input and view various information. For example, the terminal apparatus 1 is implemented by a personal computer (PC), a tablet PC, a personal digital assistant (PDA), a mobile phone, or the like operated by the medical personnel or the subject. For example, the terminal apparatus 1 receives biometric data of the subject, and transmits the received biometric data to the data server 2 or the medical information processing apparatus 3. The terminal apparatus 1 also receives processing results from the medical information processing apparatus 3, and displays the received processing results on a display of the terminal apparatus 1. The display of the terminal apparatus 1 is an example of a display. The processing results are described in detail below.

The data server 2 stores the biometric data of the subject. Specifically, the data server 2 receives the biometric data from the terminal apparatus 1 via the network 4, and stores the biometric data in storage circuitry of the data server 2. For example, the data server 2 stores biometric data including the subject's age, height, and weight, blood test results including the stem cell content, biomarker analysis results, and the like.

The medical information processing apparatus 3 acquires various information from each apparatus in the medical support system 100, and performs various information processes by using the acquired information. Specifically, the medical information processing apparatus 3 acquires the biometric data of the subject and generates information on the probability of success in cell production in the future based on biological sample of the subject, on the basis of the acquired biometric data. For example, the medical information processing apparatus 3 is implemented by computer equipment such as servers and workstations.

The medical information processing apparatus 3 is managed by a management company that provides regenerative medicine services, and includes, for example, a communication interface 31, an input interface 32, a display 33, storage circuitry 34, and processing circuitry 35.

The communication interface 31 controls the transmission and communication of various data transmitted and received between the medical information processing apparatus 3 and external apparatuses (the terminal apparatus 1, the data server 2, and the like) connected via the network 4. Specifically, the communication interface 31 is connected to the processing circuitry 35, and transmits data received from the external apparatus to the processing circuitry 35 or transmits data received from the processing circuitry 35 to the external apparatus. For example, the communication interface 31 is implemented by a network card, a network adapter, a network interface controller (NIC), or the like.

The input interface 32 receives input operations for various instructions and various information from an operator. Specifically, the input interface 32 is connected to the processing circuitry 35, and converts the input operations received from the operator into electrical signals and transmits the electrical signals to the processing circuitry 35. For example, the input interface 32 is implemented by a trackball, a switch button, a mouse, a keyboard, a touchpad for performing input operations by touching an operation surface, a touchscreen that integrates a display screen and a touchpad, a non-contact input interface using an optical sensor, a voice input interface, and the like. In the present specification, the input interface 32 is not limited only to those with physical operating components such as a mouse and a keyboard. For example, an example of the input interface 32 also includes electrical signal processing circuitry that receives electrical signals corresponding to input operations from an external input device installed separately from the device and transmits the electrical signals to control circuitry.

The display 33 displays various information and various data. Specifically, the display 33 is connected to the processing circuitry 35 and displays various information and various data received from the processing circuitry 35. For example, the display 33 displays the information on the probability of success in cell production in the future. For example, the display 33 is implemented by a liquid crystal display, a cathode ray tube (CRT) display, a touch panel, or the like. The display 33 is an example of a display.

The storage circuitry 34 stores various data and various computer programs. Specifically, the storage circuitry 34 is connected to the processing circuitry 35, and stores data received from the processing circuitry 35 or reads stored data and transmits the read data to the processing circuitry 35. For example, the storage circuitry 34 is implemented by a semiconductor memory element such as a read only memory (ROM), a random access memory (RAM), and a flash memory, a hard disk, an optical disk, or the like. Note that the storage circuitry 34 may be implemented by a cloud computer connected to the medical information processing apparatus 3 via the network 4.

The processing circuitry 35 controls the entire medical information processing apparatus 3. For example, the processing circuitry 35 performs various processes in response to input operations received from the operator via the input interface 32. For example, the processing circuitry 35 receives data transmitted from the external apparatus via the communication interface 31, and stores the received data in the storage circuitry 34. For example, the processing circuitry 35 transmits data received from the storage circuitry 34 to the communication interface 31, thereby transmitting the data to the external apparatus. For example, the processing circuitry 35 displays the data received from the storage circuitry 34 on the display 33. The processing circuitry 35 also performs a process of generating and displaying the information on the probability of success in cell production in the future based on the biological samples of the subject.

For example, as illustrated in FIG. 1, the processing circuitry 35 performs a control function 351, a data acquisition function 352, a generation function 353, and an output function 354. The data acquisition function 352 is an example of a data acquisition unit. The generation function 353 is an example of a generation unit. The output function 354 is an example of an output unit.

The control function 351 controls various graphical user interface (GUI) and various display information to be displayed on the display 33 in response to operations input via the input interface 32. For example, the control function 351 causes the display 33 to display information generated by the generation function 352

The data acquisition function 352 acquires the biometric data of the subject to be examined or treated. Note that the process performed by the data acquisition function 352 is described in detail below.

The generation function 353 generates the information on the probability of success in cell production in the future based on the biological samples of the subject, on the basis of the biometric data. Note that the process performed by the generation function 353 is described in detail below.

The output function 354 outputs the information on the probability of success in cell production in the future to the terminal apparatus 1 (first terminal) via the network 4. Note that the process performed by the output function 354 is described in detail below.

The processing circuitry 35 described above is implemented by, for example, a processor. In such a case, the respective processing functions described above are stored in the storage circuitry 34 in the form of computer programs executable by a computer. The processing circuitry 35 reads the computer programs stored in the storage circuitry 34 and executes the read computer programs, thereby implementing functions corresponding to the executed computer programs. In other words, the processing circuitry 35 in the state of reading the computer programs has each of the processing functions illustrated in FIG. 1.

Note that the processing circuitry 35 may be configured by combining a plurality of independent processors, and respective processors may implement respective processing functions by executing respective computer programs. The processing functions of the processing circuitry 35 may be implemented by being appropriately distributed or integrated into single processing circuitry or a plurality of pieces of processing circuitry. Each processing function of the processing circuitry 35 may be implemented by a mixture of hardware such as circuits and software. Although an example in which the computer program corresponding to each processing function is stored in the single storage circuitry 34 is described above, the embodiment is not limited thereto. For example, a computer program corresponding to each processing function may be distributed and stored in a plurality of pieces of storage circuitry, and the processing circuitry 35 may be configured to read each computer program from each storage circuitry and execute the read computer program. Note that a part of each processing function of the processing circuitry 35 may be implemented by a cloud computer connected to the medical information processing apparatus 3 via the network 4.

The above is an example of the configuration of the medical support system 100 according to the present embodiment. Under such a configuration, the medical support system 100 provides regenerative medicine that responds to individual differences. As described above, autologous transplantation is being considered as one solution for avoiding the risk of immune rejection in regenerative medicine; however, since there are always individual differences in cells for each subject (patient) for regenerative medicine, in order to provide regenerative medicine widely, it is necessary to absorb these individual differences and reliably establish and differentiate necessary cells, organs, and the like for provision. However, at present, the biological mechanisms of establishment and differentiation of cells, organs, and the like remain unclear, making it difficult to absorb a wide range of individual differences and provide autologous transplantation with appropriate quality.

Therefore, the medical support system 100 according to the present embodiment generates information on the probability of success in cell production in the future of a subject on the basis of biometric data of the subject, thereby providing regenerative medicine that responds to individual differences. The medical support system 100 according to the present embodiment generates information on the probability of success in iPS cell establishment as the information on the probability of success in cell production.

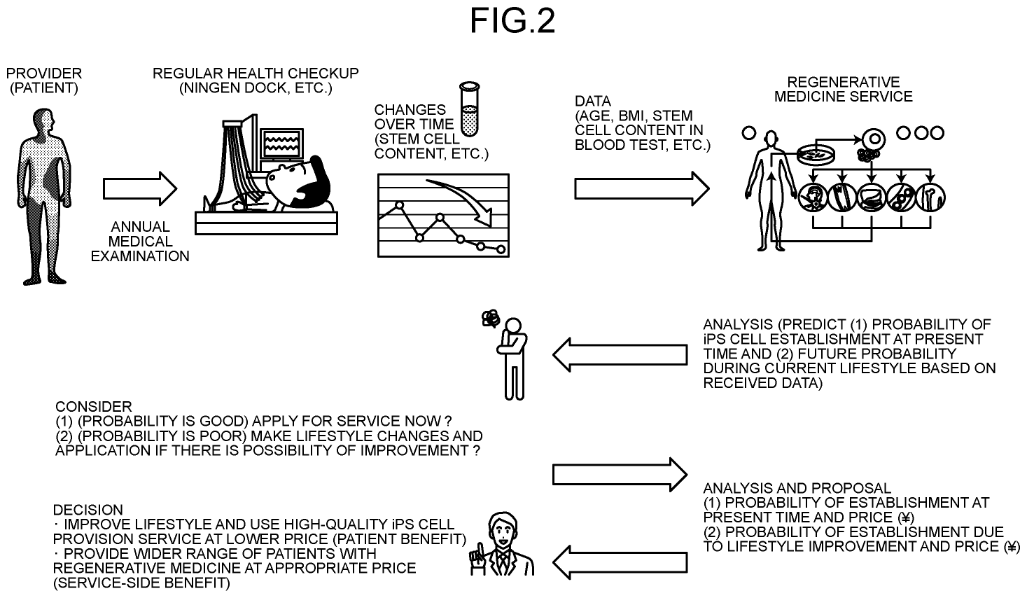

An overview of the medical support system 100 according to the first embodiment is described below with reference to FIG. 2. FIG. 2 is a diagram for explaining an overview of the medical support system 100 according to the first embodiment. For example, as illustrated in FIG. 2, the medical support system 100 acquires biometric data from a provider (patient) who has authorized to provide his/her biometric data, and performs analysis based on the biometric data.

The provider is a person who is a subject to be examined or treated and who is considering receiving regenerative medicine services (here, iPS cell provision services). Such a provider, for example, provides the results of a regular health checkup (ningen dock or the like) received annually to the medical support system 100 as biometric data. The ningen dock is comprehensive health checkup system. As illustrated in FIG. 2, the biometric data includes, for example, age, body mass index (BMI), stem cell content in a blood test, and the like. The BMI or the stem cell content also includes information on changes over time based on annual results.

In the medical support system 100, the medical information processing apparatus 3 acquires the above biometric data from the terminal apparatus 1 or the data server 2, and performs analysis based on the acquired biometric data. Specifically, as illustrated in FIG. 2, the medical information processing apparatus 3 predicts the probability of iPS cell establishment at the present time, and the probability of iPS cell establishment in the future when the current lifestyle is continued.

This allows the provider to consider applying for the service now when the probability of iPS cell establishment at the present time is good. When the probability of iPS cell establishment at the present time is poor, the provider can consider whether the probability of iPS cell establishment can be improved due to lifestyle improvements on the basis of the probability of iPS cell establishment in the future when the current lifestyle is continued, and when there is a possibility of improvement, the provider can consider improving the lifestyle and applying for the service.

On the basis of the probability of iPS cell establishment, the medical information processing apparatus 3 can also calculate and present the cost related to iPS cell production. Specifically, the medical information processing apparatus 3 presents the provider with the probability of iPS cell establishment at the present time and the cost thereof, and the probability of iPS cell establishment and the cost thereof when lifestyle is improved.

The provider makes the final decision by confirming the presented information (that is, the probability of iPS cell establishment and the cost thereof). In general, the higher the probability of iPS cell establishment, the lower the cost related to iPS cell production. Consequently, by making the final decision based on the presented information, the provider can benefit from high-quality iPS cell provision services at a lower price. Management companies that provide iPS cell provision services can provide regenerative medicine that responds to individual differences to a wider range of patients at an appropriate price.

As described above, by presenting the actual probability of iPS cell establishment, it is possible to increase the motivation to use the service not only for patients with a high awareness of health anxiety, but also for younger subjects with a low awareness of health anxiety. In general, younger people have a higher probability of establishing iPS cells and producing good iPS cells, but they are less motivated to use regenerative medicine services (iPS cell provision services) due to their lower awareness of health anxiety. However, by presenting these young people with their probabilities of iPS cell establishment at the present time and in the future, it is possible to gain their understanding of the iPS cell provision services and encourage them to use the services.

When the probability of iPS cell establishment is improved due to lifestyle improvements, the fee for the regenerative medicine services may be discounted to motivate people to improve their constitutions and use the services.

The above is an overview of the medical support system 100 according to the first embodiment. Details of processes performed by the medical support system 100 according to the first embodiment are described below.

### Process of Acquiring Biometric Data

The data acquisition function 352 according to the first embodiment acquires biometric data of a subject in response to an application of the subject for the analysis of the probability of iPS cell establishment. For example, upon receiving an application for the analysis of the probability of iPS cell establishment from the terminal apparatus 1 operated by a medical personnel or the subject himself/herself, the data acquisition function 352 acquires the biometric data of the subject from the terminal apparatus 1 or the data server 2.

The biometric data acquired by the data acquisition function 352 includes at least one of the age, height, and weight of the subject and the stem cell content in the blood, or data based on the at least one. For example, the biometric data includes the age, BMI, and stem cell content in the blood of the subject. Note that the above example of the biometric data is merely an example and may include other information (for example, blood cholesterol level and the like).

The data acquisition function 352 acquires the above biometric data collected regularly from the subject. That is, the data acquisition function 352 acquires biometric data of time series. This allows the data acquisition function 352 to acquire information on, for example, changes in BMI over time and changes in stem cell content over time.

### Process of Estimating Probability of iPS Cell Establishment at Present Time

The generation function 353 according to the first embodiment generates information on the probability of success in cell production. The information on the probability of success in cell production includes information on the probability of cell establishment, and is information on the probability of iPS cell establishment in the present embodiment.

On the basis of the biometric data acquired by the data acquisition function 352, the generation function 353 generates information on the probability of success in cell production at present. Specifically, on the basis of the correlation between the stem cell content in the blood and the probability of cell establishment, the generation function 353 generates the information on the probability of cell establishment from the stem cell content in the blood of the subject included in the biometric data. That is, on the basis of the correlation between the stem cell content and the probability of iPS cell establishment, the generation function 353 estimates the probability of iPS cell establishment for the subject from the stem cell content in the blood of the subject.

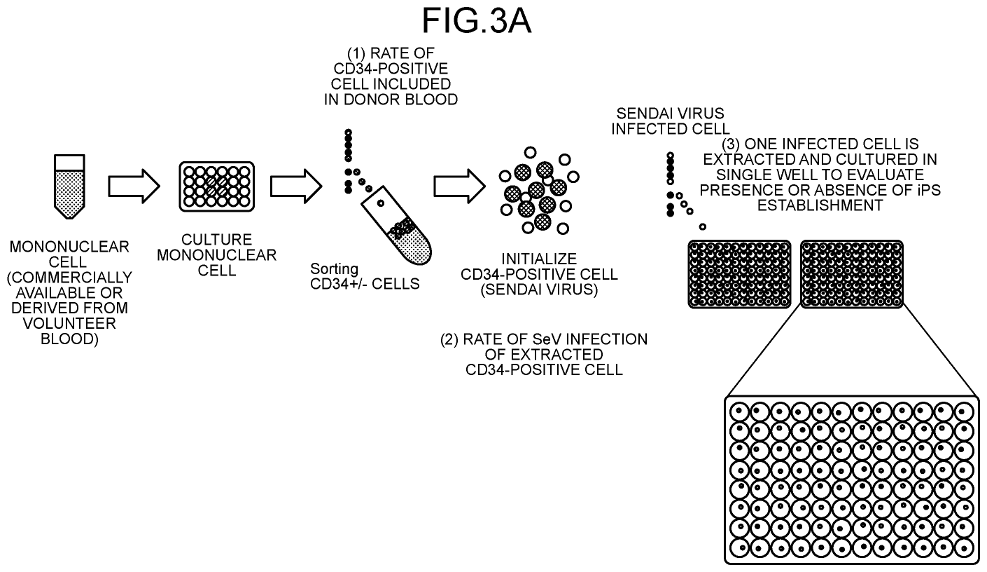

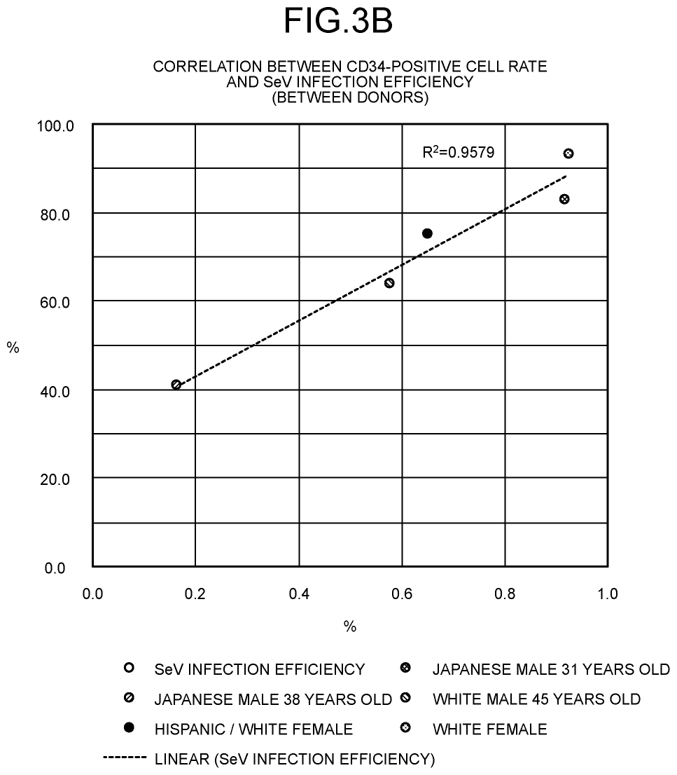

FIGS. 3A and 3B are diagrams for explaining the correlation between the stem cell content and the probability of iPS cell establishment according to the first embodiment. FIG. 3A is a diagram for explaining a method for evaluating the correlation between the stem cell content and the probability of iPS cell establishment. FIG. 3B also illustrates a graph with the probability of iPS cell establishment on a vertical axis and the stem cell content on a horizontal axis.

Results when the correlation between the stem cell content and the probability of iPS cell establishment is evaluated by the relation between a CD34-positive cell rate and the infection efficiency of Sendai virus (SeV) are described below. As illustrated in FIG. 3A, in this evaluation, mononuclear cells (commercially available or derived from volunteer blood) are first cultured to acquire the percentage of CD34-positive cells in the cultured cells (CD34-positive cell rate). Note that the CD34-positive cells are cells (that is, stem cells) that are positive for CD34 being a marker (signal) emitted by stem cells. Consequently, the CD34-positive cell rate indicates the content of the stem cells in the cultured cells.

Subsequently, the extracted CD34-positive cells are infected with Sendai virus, and an initialization factors for producing iPS cells are introduced into the CD34-positive cells. Subsequently, the presence or absence of Sendai virus infection is evaluated by the presence or absence of green fluorescent protein (GFP) positivity in the cells, and Sendai virus-infected cells are cultured one by one in a single well to evaluate the presence or absence of the iPS cell establishment.

FIG. 3B illustrates a graph when the CD34-positive cell rate is used as the stem cell content and the Sendai virus infection efficiency (the rate of Sendai virus infection on the CD34-positive cells) is used as the probability of iPS cell establishment. FIG. 3B also illustrates a graph in which the CD34-positive cell rate and the Sendai virus infection efficiency are acquired for five individuals of different races, genders, and ages and the acquired values are plotted. As illustrated in FIG. 3B, since there is a correlation between the CD34-positive cell rate and the Sendai virus infection efficiency, it can be seen that the probability of iPS cell establishment can be estimated from the stem cell content. The graph illustrated in FIG. 3B is merely an example.

For example, the generation function 353 estimates the probability of iPS cell establishment in a subject on the basis of the correlation between the CD34-positive cell rate (stem cell content) and the Sendai virus infection efficiency (probability of iPS cell establishment) illustrated in FIG. 3B. In one example, the generation function 353 extracts the numerical value of the CD34-positive cell rate at the present time from biometric data of the subject. Subsequently, on the basis of the correlation between the CD34-positive cell rate and the Sendai virus infection efficiency, the generation function 353 estimates the numerical value of the Sendai virus infection efficiency from the extracted numerical value of the CD34-positive cell rate. In this case, the stem cell content included in the biometric data acquired by the data acquisition function 352 is the CD34-positive cell rate.

In this way, the generation function 353 estimates the probability of iPS cell establishment in the subject by estimating the Sendai virus infection efficiency from the numerical value of the CD34-positive cell rate of the subject. Process of Estimating Probability of iPS Cell Establishment in Future

The generation function 353 generates information on the probability of success in cell production in the future on the basis of biometric data of time series. Specifically, the generation function 353 estimates the stem cell content in the future on the basis of changes in the stem cell content in the biometric data of time series, and generates information on the probability of success in cell production in the future on the basis of the estimated stem cell content.

For example, the generation function 353 acquires changes in the CD34-positive cell rate over time from the biometric data of time series acquired by the data acquisition function 352. Subsequently, on the basis of the trend of changes in the acquired CD34-positive cell rate over time, the generation function 353 estimates the numerical value of the CD34-positive cell rate in the future (after a predetermined period of time). On the basis of the correlation between the CD34-positive cell rate and the Sendai virus infection efficiency described above, the generation function 353 further estimates the numerical value of the Sendai virus infection efficiency from the estimated numerical value of the CD34-positive cell rate. Thus, the generation function 353 estimates the probability of iPS cell establishment in the future (after a predetermined period of time).

The generation function 353 further generates information on the probability of success in cell production in the future when lifestyle changes are made. Specifically, the generation function 353 estimates the stem cell content in the future from changes in the subject's BMI and stem cell content, and generates information on the probability of success in cell production in the future on the basis of the estimated stem cell content.

For example, the generation function 353 acquires changes in the CD34-positive cell rate over time and changes in BMI over time from the biometric data of time series acquired by the data acquisition function 352. Subsequently, the generation function 353 estimates a CD34-positive cell rate when the subject's BMI changes from the correspondence between the CD34-positive cell rate and the BMI value. In one example, when a subject, who has the BMI value out of a standard range, has changed his/her lifestyle to bring his/her BMI value within the standard range, the generation function 353 estimates a CD34-positive cell rate. Process of Estimating Optimal Time of iPS Cell Establishment

The generation function 353 further generates information on the establishment time of cells on the basis of the information on the probability of cell establishment and a storage period the cells. Specifically, on the basis of the estimated probability of iPS cell establishment and the aging of iPS cells during the storage period, the generation function 353 estimates an optimal time for establishing the iPS cells for the subject.

For example, the generation function 353 calculates the period from the subject's age to the age at which regenerative medicine is applied, and estimates the time within the calculated period for which estimating iPS cells is optimal. Note that the age at which regenerative medicine is applied is set on the basis of the subject's medical history and the average of patients who have used regenerative medicine services, and the like.

In such a case, the generation function 353 estimates how the subject's probability of iPS cell establishment changes during the above period (future changes over time). Subsequently, on the basis of the future changes over time of the estimated probability of iPS cell establishment and the correspondence between the storage period of the iPS cells and deterioration (for example, probability of failure of differentiation), the generation function 353 estimates an optimal time for establishing the iPS cells for the subject. For example, when the degradation corresponding to each storage period is taken into account for each probability of iPS cell establishment at each time of the above period, the generation function 353 estimates, as an optimal time, the time that has the least impact on successful differentiation. In an example, the generation function 353 estimates, as an optimal time, the time when the number of cells that successfully differentiate is highest.

### Process of Generating Display Information

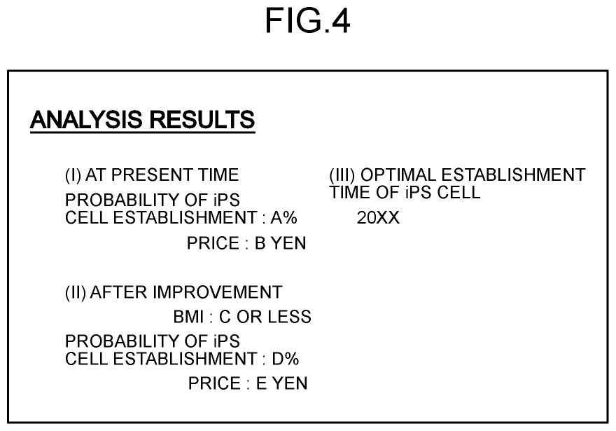

The generation function 353 generates display information on the estimated information. Specifically, the generation function 353 generates display information including the probability of iPS cell establishment for the subject. FIG. 4 is a diagram illustrating an example of display information according to the first embodiment. For example, as illustrated in FIG. 4, the generation function 353 generates display information for displaying analysis results including (I) the probability of iPS cell establishment at the present time, (II) the probability of iPS cell establishment after lifestyle improvements, and (III) the optimal establishment time of iPS cells.

(I) The present time can include “price: B yen” for the establishment of iPS cells at the present time, in addition to “probability of iPS cell establishment: A %”. (II) The post-improvement can also include the value of BMI “C or less” after improvement and “Price: E yen” when iPS cells are established after improvement, in addition to “probability of iPS cell establishment: D%”. Note that the information on the cost per probability of iPS cell establishment is stored in the storage circuitry 34 in advance, and the generation function 353 acquires the price information illustrated in FIG. 4 by reading the information from the storage circuitry 34.

### Process of Displaying Display Information

The control function 351 causes the display 33 to display the display information generated by the generation function 353. For example, the control function 351 causes the display 33 to display the information illustrated in FIG. 4. That is, the display 33 displays information on the probability of success in cell production in the future. The display 33 further displays information on the probability of success in cell production in the future when lifestyle changes are made. The display 33 also displays information on the cost required for cell production in the future together with the information on the probability of success in cell production in the future.

### Process of Outputting Information

The output function 354 outputs the information (for example, display information) generated by the generation function 353 to other apparatuses connected to the network 4 via the communication interface 31. For example, the output function 354 outputs the information illustrated in FIG. 4 to the terminal apparatus 1. That is, the medical support system 100 can also provide an analysis service that receives analysis requests from other apparatuses via the network 4 and transmits the analysis results.

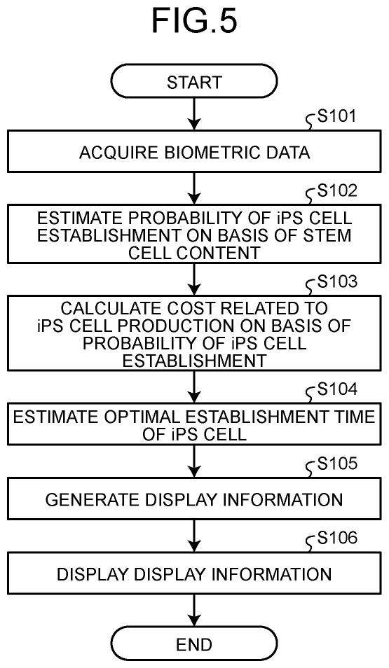

The processing procedure performed by the medical information processing apparatus 3 is described below with reference to FIG. 5. FIG. 5 is a flowchart showing an example of the processing procedure of the medical information processing apparatus 3 according to the first embodiment.

For example, as illustrated in FIG. 5, in the present embodiment, the data acquisition function 352 acquires biometric data of a subject from the terminal apparatus 1 or the data server 2 (step S101). This process is implemented, for example, by the processing circuitry 35 calling a computer program corresponding to the data acquisition function 352 from the storage circuitry 34 and executing the called computer program.

Subsequently, the generation function 353 estimates the probability of iPS cell establishment on the basis of the stem cell content in the acquired biometric data (step S102). The generation function 353 estimates the probability of iPS cell establishment at the present time and the probability of iPS cell establishment in the future, respectively. This process is implemented, for example, by the processing circuitry 35 calling a computer program corresponding to the generation function 353 from the storage circuitry 34 and executing the called computer program.

Subsequently, the generation function 353 calculates the cost related to iPS cell production on the basis of the probability of iPS cell establishment (step S103). This process is implemented, for example, by the processing circuitry 35 calling a computer program corresponding to the generation function 353 from the storage circuitry 34 and executing the called computer program.

Subsequently, the generation function 353 estimates the optimal establishment time of iPS cells (step 5104) and generates display information (step S105). This process is implemented, for example, by the processing circuitry 35 calling a computer program corresponding to the generation function 353 from the storage circuitry 34 and executing the called computer program.

Subsequently, the control function 351 causes the display 33 to display the display information (Step S106). This process is implemented, for example, by the processing circuitry 35 calling a computer program corresponding to the control function 351 from the storage circuitry 34 and executing the called computer program.

In FIG. 5, the processing procedure including the estimation of the optimal establishment time of iPS cells is described; however, the processing procedure may not include the estimation of the optimal establishment time of iPS cells. In FIG. 5, the processing procedure including the calculation of the cost related to iPS cell production is described; however, the processing procedure may not include the calculation of the cost related to iPS cell production. In FIG. 5, the process procedure for estimating the probability of iPS cell establishment at the present time and the probability of iPS cell establishment in the future is described; however, only the probability of iPS cell establishment in the future may be estimated.

As described above, according to the first embodiment, the data acquisition function 352 acquires biometric data of a subject to be examined or treated. The generation function 353 generates the information on the probability of success in cell production in the future based on biological samples of the subject, on the basis of the biometric data. The display 33 displays information on the probability of success in cell production in the future. Consequently, the medical support system 100 according to the first embodiment can acquire the probability of success in cell production in the future for each subject, and makes it possible to provide regenerative medicine that responds to individual differences in each subject.

According to the first embodiment, the data acquisition function 352 acquires biometric data of time series. The generation function 353 generates information on the probability of success in cell production in the future on the basis of the biometric data of time series. Consequently, the medical support system 100 according to the first embodiment makes it possible to acquire the probability of success in cell production in the future according to the propensity of the subject.

According to the first embodiment, the information on the probability of success in cell production includes information on the probability of cell establishment. Consequently, the medical support system 100 according to the first embodiment makes it possible to provide information on the cell establishment that responds to individual differences.

According to the first embodiment, the biometric data includes the stem cell content in the blood of the subject. According to the first embodiment, on the basis of the correlation between the stem cell content in the blood and the probability of cell establishment, the generation function 353 generates information on the probability of cell establishment from the stem cell content in the blood of the subject included in the biometric data. Consequently, the medical support system 100 according to the first embodiment makes it possible to easily estimate the information on the probability of cell establishment.

According to the first embodiment, the generation function 353 further generates information on the probability of success in cell production at present. Consequently, the medical support system 100 according to the first embodiment can acquire the probability of success in cell production at the present time for each subject, and makes it possible to provide regenerative medicine that is more responsive to individual differences in each subject.

According to the first embodiment, the generation function 353 further generates information on the probability of success in cell production in the future when the lifestyle changes are made. The display 33 further displays the information on the probability of success in cell production in the future when the lifestyle changes are made. Consequently, the medical support system 100 according to the first embodiment makes it possible to present the possibility of improving the probability of success in cell production in the future.

According to the first embodiment, the biometric data includes at least one of the age, height, and weight of the subject and the stem cell content in the blood, or data based on the at least one. Consequently, the medical support system 100 according to the first embodiment makes it possible to acquire information indicating the characteristics of each subject.

According to the first embodiment, the generation function 353 estimates the stem cell content in the future from changes in the subject's BMI and stem cell content, and generates information on the probability of success in cell production in the future on the basis of the estimated stem cell content. Consequently, the medical support system 100 according to the first embodiment makes it possible to easily estimate changes in the stem cell content when the lifestyle is improved.

According to the first embodiment, the display 33 displays information on the cost related to cell production in the future together with information on the probability of success in cell production in the future. Consequently, the medical support system 100 according to the first embodiment makes it possible to provide regenerative medicine services at an appropriate price.

According to the first embodiment, the generation function 353 further generates information on the establishment time of cells on the basis of information on the probability of cell establishment and a storage period the cells. Consequently, the medical support system 100 according to the first embodiment makes it possible to use regenerative medicine services at the appropriate timing.

According to the first embodiment, information on the probability of success in the production of living cells includes information on the probability of iPS cell establishment. Consequently, the medical support system 100 according to the first embodiment makes it possible to provide the probability of iPS cell establishment.

### Second Embodiment

The first embodiment described above describes a case in which information on the probability of success in iPS cell establishment is generated as information on the probability of success in cell production. The second embodiment describes a case in which information on the probability of cell differentiation is generated as the information on the probability of success in cell production. Note that the second embodiment differs from the first embodiment in processes performed by the data acquisition function 352 and processes performed by the generation function 353. These are mainly described below.

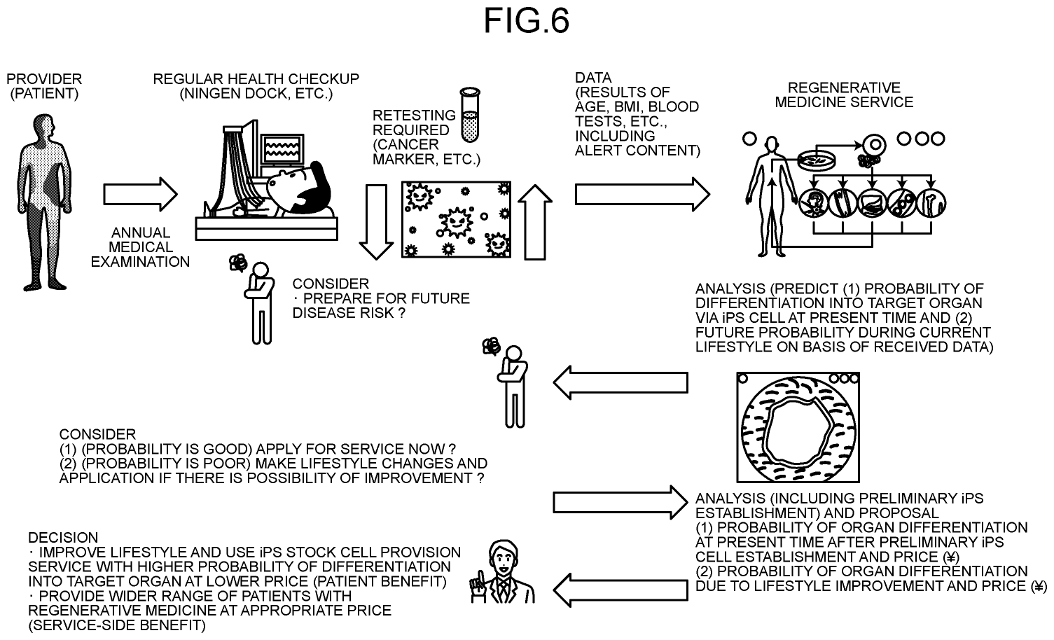

First, an overview of a medical support system 100 according to the second embodiment is described with reference to FIG. 6. FIG. 6 is a diagram for explaining an overview of the medical support system 100 according to the second embodiment. For example, as illustrated in FIG. 6, the medical support system 100 according to the second embodiment acquires biometric data from a provider (patient) who has authorized to provide his/her biometric data, and performs analysis based on the biometric data.

As illustrated in FIG. 6, the biometric data according to the second embodiment is, for example, results of age, BMI, stem cell content in blood tests, and the like, including alert content. The BMI or the stem cell content also includes information on changes over time based on annual results. The alert content is test results that require retesting (for example, positive results for cancer markers such as CEA and CA-19-9 for gastric cancer and colon cancer).

In the medical support system 100 according to the second embodiment, the medical information processing apparatus 3 acquires the above biometric data from the terminal apparatus 1 or the data server 2 and performs analysis based on the acquired biometric data. Specifically, as illustrated in FIG. 6, the medical information processing apparatus 3 predicts the current probability, and the future probability when the current life is continued, with respect to the probability of differentiation from iPS cells to a target organ corresponding to the alert content.

This allows the provider to consider applying for organ differentiation services now when the probability of differentiation at the present time is good. When the probability of differentiation at the present time is poor, the provider can consider whether the probability of differentiation can be improved due to lifestyle improvements on the basis of the probability of differentiation in the future when the current lifestyle is continued, and when there is a possibility of improvement, the provider can consider improving the lifestyle and applying for the service.

The medical information processing apparatus 3 can also calculate and present the cost related to organ differentiation on the basis of the probability of differentiation from the iPS cells to the target organ. Specifically, the medical information processing apparatus 3 presents the provider with the probability of differentiation at the present time and the cost thereof, as well as the probability of differentiation and the cost thereof when the lifestyle is improved.

The provider confirms the presented information (probability of differentiation and the cost thereof) and makes the final decision. In general, the differentiation of iPS cells into organs requires a long period of time and a large amount of cost. In a case of a lower probability of differentiation, more cost is needed. Consequently, by making the final decision based on the presented information, the provider can benefit from a service with a higher probability of differentiation at a lower price. Management companies that provide the service can provide regenerative medicine that responds to individual differences to a wider range of patients at an appropriate price.

The above is an overview of the medical support system 100 according to the second embodiment. Details of processes performed by the medical support system 100 according to the second embodiment are described below.

### Process of Acquiring Biometric Data

The data acquisition function 352 according to the second embodiment acquires biometric data of a subject in response to an application of the subject for the analysis of the probability of differentiation from iPS cells to the target organ. For example, upon receiving an application for the analysis of the probability of differentiation from the terminal apparatus 1 operated by a medical personnel or the subject himself/herself, the data acquisition function 352 acquires the biometric data of the subject from the terminal apparatus 1 or the data server 2. The biometric data acquired by the data acquisition function 352 includes the alert content described above.

### Process of Estimating Probability of Differentiation at Present Time

The generation function 353 according to the second embodiment specifies information indicating a site of the subject with a high disease risk and generates information on the probability of differentiation for the site with a high disease risk. Specifically, on the basis of the alert content included in the biometric data acquired by the data acquisition function 352, the generation function 353 specifies the site of the subject with a high disease risk. Subsequently, the generation function 353 estimates the probability of differentiation from the iPS cells to the identified site (target organ).

The generation function 353 generates information on the probability of cell differentiation on the basis of the characteristics of the established cells. For example, the generation function 353 acquires the characteristics of iPS cells that have been preliminarily established based on the blood of the subject, and estimates the probability of differentiation into the target organ on the basis of the acquired characteristics. That is, on the basis of the characteristics of actually established iPS cells, the generation function 353 estimates whether the iPS cells are likely to differentiate into the target organ.

The characteristics of the iPS cells may be obtained by, for example, biomarkers. For example, early in development, cells differentiate into three germ layers: endoderm, mesoderm, and ectoderm. Subsequently, the endoderm, the mesoderm, and the ectoderm are differentiated into distinct tissues and organs. Therefore, the generation function 353 acquires the analysis results of the biomarkers related to the above three germ layers performed on the actually established iPS cells, and estimates whether the iPS cells are likely to differentiate into the target organ on the basis of the acquired analysis results.

For example, when the target organ is the colon, the generation function 353 estimates the probability of differentiation on the basis of the analysis of endoderm markers for iPS cells. That is, the generation function 353 estimates a high probability of differentiation when strong expression of the endoderm markers is observed in the iPS cells. On the other hand, when the expression of the endoderm markers is weak in the iPS cells, the generation function 353 estimates that the probability of differentiation is low. Process of Estimating Probability of Differentiation in Future

The generation function 353 estimates the probability of differentiation in the future on the basis of the probability of iPS cell establishment in the future. Specifically, when the probability of iPS cell establishment in the future increases (improves), the generation function 353 estimates that the probability of differentiation in the future will increase, and when the probability of iPS cell establishment in the future decreases, the generation function 353 estimates that the probability of differentiation in the future will decrease. That is, as described in the first embodiment, the generation function 353 estimates the probability of iPS cell establishment in the future from the biometric data of time series, and estimates whether the probability of differentiation at the present time will increase or decrease on the basis of the estimated probability of establishment.

### Process of Generating Display Information

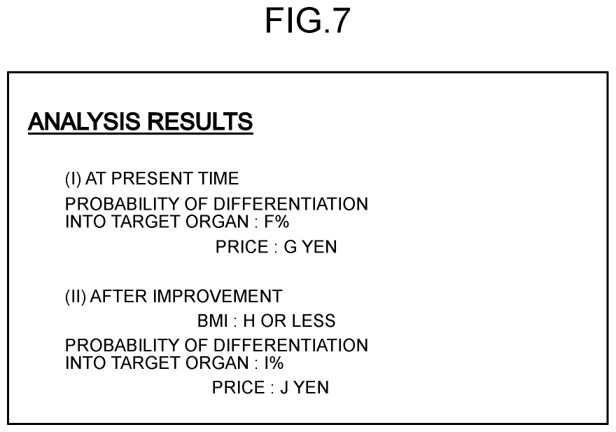

The generation function 353 generates display information including the probability of differentiation from the iPS cells to the target organ. FIG. 7 is a diagram illustrating an example of display information according to the second embodiment. For example, as illustrated in FIG. 7, the generation function 353 generates display information for displaying analysis results including (I) the probability of differentiation into the target organ at the present time and (II) the probability of differentiation into the target organ after lifestyle improvements.

(I) The present time can include “price: G yen” for differentiation into the target organ at the present time, in addition to “probability of differentiation into the target organ: F %”. (II) The post-improvement can also include the value of BMI “H or less” after improvement and “Price: J yen” when differentiated into the target organ after improvement, in addition to “probability of differentiation into the target organ: I %”. Note that information on the cost per probability of differentiation is stored in the storage circuitry 34 in advance, and the generation function 353 acquires the price information illustrated in FIG. 7 by reading the information from the storage circuitry 34.

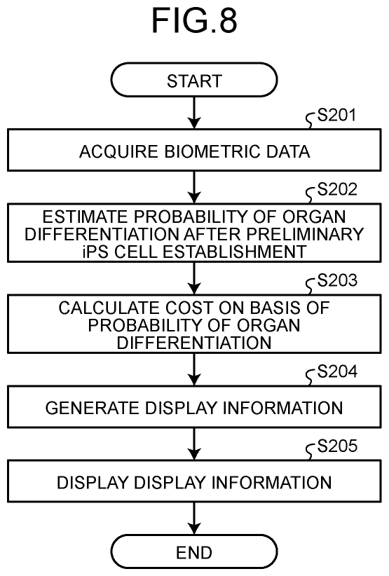

The processing procedure performed by the medical information processing apparatus 3 according to the second embodiment is described below with reference to FIG. 8. FIG. 8 is a flowchart showing an example of the processing procedure of the medical information processing apparatus 3 according to the second embodiment.

For example, as illustrated in FIG. 8, in the present embodiment, the data acquisition function 352 acquires biometric data of a subject from the terminal apparatus 1 or the data server 2 (step S201). This process is implemented, for example, by the processing circuitry 35 calling a computer program corresponding to the data acquisition function 352 from the storage circuitry 34 and executing the called computer program.

Subsequently, the generation function 353 specifies a target organ on the basis of the biometric data and estimates the probability of differentiation into the target organ after preliminary iPS cell establishment (step S202). The generation function 353 estimates the probability of differentiation at the present time and the probability of differentiation in the future, respectively. This process is implemented, for example, by the processing circuitry 35 calling a computer program corresponding to the generation function 353 from the storage circuitry 34 and executing the called computer program.

Subsequently, the generation function 353 calculates the cost related to the differentiation into the target organ on the basis of the probability of differentiation (step S203), and generates display information (step S204). This process is implemented, for example, by the processing circuitry 35 calling a computer program corresponding to the generation function 353 from the storage circuitry 34 and executing the called computer program.

Subsequently, the control function 351 causes the display 33 to display the display information (Step S205). This process is implemented, for example, by the processing circuitry 35 calling a computer program corresponding to the control function 351 from the storage circuitry 34 and executing the called computer program.

In FIG. 8, the processing procedure including the calculation of the cost related to differentiation into the target organ is described; however, the processing procedure may not include the calculation of the cost related to differentiation into the target organ.

As described above, according to the second embodiment, information on the probability of success in cell production includes information on the probability of cell differentiation. Consequently, the medical support system 100 according to the second embodiment makes it possible to provide information on the probability of differentiation that responds to individual differences.

According to the second embodiment, the generation function 353 specifies information indicating a site of a subject with a high disease risk and generates information on the probability of differentiation for the site with a high disease risk. Consequently, the medical support system 100 according to the second embodiment can estimate the probability of differentiation into different target organs for each subject, making it possible to provide regenerative medicine that responds to individual differences.

According to the second embodiment, the generation function 353 generates information on the probability of cell differentiation on the basis of the characteristics of established cells. Consequently, the medical support system 100 according to the second embodiment makes it possible to estimate the probability of cell differentiation.

### Other Embodiments

The above-described embodiments describe a case in which improvement in a subject's constitution is determined by BMI. However, embodiments are not limited thereto and for example, an index such as blood cholesterol may be used.

In the embodiments described above, cases in which the data acquisition unit, the generation unit, and the output unit in the present specification are implemented by the data acquisition function, the generation function, and the output function of the processing circuitry, respectively, are described as examples; however, embodiments are not limited thereto. For example, the data acquisition unit, the generation unit, and the output unit in the present specification may be implemented by the data acquisition function, the generation function, and the output function described in the embodiments, or the same functions may also be implemented by hardware only, software only, or a combination of hardware and software.

The term “processor” used in the description of the above-described embodiments means, for example, circuitry such as a central processing unit (CPU), a graphics processing unit (GPU), an application specific integrated circuit (ASIC), a programmable logic device (for example, a simple programmable logic device (SPLD)), a complex programmable logic device (CPLD), and a field programmable gate array (FPGA)). Instead of storing computer programs in the storage circuitry, the computer programs can be configured to be incorporated directly into the circuitry of the processor. In this case, the processor implements the functions by reading and executing the computer programs incorporated in the circuitry. Each processor of the present embodiment is not limited to being configured as single circuitry for each processor, and one processor may be configured by combining a plurality of pieces of independent circuitry to implement the functions thereof.

The medical information processing program to be executed by the processor is provided by being incorporated in advance in a read only memory (ROM), storage circuity, or the like. Note that the medical information processing program may be provided by being recorded on a computer readable non-transitory storage medium, such as a CD (compact disc)-ROM, a flexible disk (FD), a CD-R (recordable), and a digital versatile disc (DVD), in a file format installable or executable in these devices. The medical information processing program may also be provided or distributed by being stored on a computer connected to a network such as the Internet and downloaded over the network. For example, the medical information processing program is configured as a module including the aforementioned each processing function. As actual hardware, the CPU reads and executes the medical information processing program from the storage medium such as ROM, and each module is loaded onto a main storage device and generated on the main storage device.

In the embodiments and variations described above, each component of each apparatus illustrated in the drawings is a functional concept, and does not necessarily have to be physically configured as illustrated in the drawings. That is, the specific form of dispersion or integration of each apparatus is not limited to that illustrated in the drawings, but can be configured by functionally or physically dispersing or integrating all or part thereof in arbitrary units according to various loads and usage conditions. Moreover, each processing function performed by each apparatus can be implemented in whole or in part by a CPU and a computer program that is analyzed and executed by the CPU, or by hardware using wired logic.

Of the processes described in the above embodiments and variations, all or part of the processes described as being performed automatically can be performed manually, or all or part of the processes described as being performed manually can be performed automatically by known methods. Other information including processing procedures, control procedures, specific names, and various data and parameters shown in the above documents and drawings may be changed as desired, unless otherwise noted.

At least one of the embodiments described above can provide regenerative medicine that responds to individual differences.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical support system comprising:
processing circuitry configured to
acquire biometric data of a subject to be examined or treated, and
generate information on a probability of success in cell production in the future based on biological samples of the subject, on the basis of the biometric data; and
a display configured to display the information on the probability of success in cell production in the future.

2. The medical support system according to claim 1, wherein
the processing circuitry is configured to
acquire the biometric data of time series, and
generate the information on the probability of success in cell production in the future on the basis of the biometric data of time series.

3. The medical support system according to claim 1, wherein the information on the probability of success in cell production includes information on a probability of cell establishment.

4. The medical support system according to claim 3, wherein the biometric data includes stem cell content in blood of the subject.

5. The medical support system according to claim 4, wherein, on the basis of a correlation between the stem cell content in the blood and the probability of cell establishment, the processing circuitry is configured to generate the information on the probability of cell establishment from the stem cell content in the blood of the subject included in the biometric data.

6. The medical support system according to claim 1, wherein the information on the probability of success in cell production includes information on a probability of cell differentiation.

7. The medical support system according to claim 6, wherein the processing circuitry is configured to specify information indicating a site of the subject with a high disease risk and generate the information on the probability of differentiation for the site with a high disease risk.

8. The medical support system according to claim 6, wherein the processing circuitry is configured to generate the information on the probability of cell differentiation on the basis of characteristics of established cells.

9. The medical support system according to claim 1, wherein the processing circuitry is further configured to generate information on a probability of success in cell production at present.

10. The medical support system according to claim 1, wherein
the processing circuitry is further configured to generate information on a probability of success in cell production in the future when lifestyle changes are made, and
the display is further configured to display the information on the probability of success in cell production in the future when the lifestyle changes are made.

11. The medical support system according to claim 1, wherein the biometric data includes at least one of age, height, and weight of the subject and stem cell content in blood, or data based on the at least one.

12. The medical support system according to claim 2, wherein the processing circuitry is configured to estimate stem cell content in the future from changes in a body mass index (BMI) and stem cell content of the subject, and generate the information on the probability of success in cell production in the future on the basis of the estimated stem cell content.

13. The medical support system according to claim 1, wherein the display is configured to display information on cost required for the cell production in the future together with the information on the probability of success in cell production in the future.

14. The medical support system according to claim 1, wherein the processing circuitry is further configured to generate information on an establishment time of cells on the basis of information on a probability of cell establishment and a storage period the cells.

15. The medical support system according to claim 1, wherein the information on the probability of success in cell production includes information on a probability of induced pluripotent stem (iPS) cell establishment.

16. A medical support method comprising:
acquiring biometric data of a subject to be examined or treated;
generating information on a probability of success in cell production in the future based on biological samples of the subject, on the basis of the biometric data; and
displaying the information on the probability of success in cell production in the future.

17. A medical information processing apparatus that is able to communicate with, via a network, a first terminal used by a medical personnel or a subject to be examined or treated, the medical information processing apparatus comprising:
processing circuitry configured to
acquire biometric data of the subject from the first terminal or another apparatus,
generate information on a probability of success in cell production in the future based on biological samples of the subject, on the basis of the biometric data, and
output the information on the probability of success in cell production in the future to the first terminal via the network.

18. A medical information processing apparatus comprising
processing circuitry configured to
acquire biometric data of a subject to be examined or treated, and
generate information on a probability of success in cell production in the future based on biological samples of the subject, on the basis of the biometric data.

19. A non-transitory computer readable storage medium including programmed instructions, wherein the instructions, when executed by a computer, cause the computer to perform:
generating, on the basis of biometric data of a subject to be examined or treated, information on a probability of success in cell production in the future based on biological samples of the subject; and
outputting the information on the probability of success in cell production in the future.
